(19) United States
(12) Patent Application Publication
(10) Pub. No.: US 2021/0040014 A1
(43) Pub. Date: Feb. 11, 2021

(54) MULTISTAGE ALKYLATION OF ISOPARAFFIN

(71) Applicant: ExxonMobil Research and Engineering Company, Annandale, NJ (US)

(72) Inventors: Vinit Choudhary, Cypress, TX (US); Doron Levin, Highland Park, NJ (US); Matthew S. Mettler, Tomball, TX (US); Joshua W. Allen, Branchburg, NJ (US)

(21) Appl. No.: 16/928,877

(22) Filed: Jul. 14, 2020

Related U.S. Application Data
(60) Provisional application No. 62/883,267, filed on Aug. 6, 2019.

(51) Int. Cl. C07C 2/76; B01J 29/70; C10L 10/10; C10L 1/16

(52) U.S. Cl. CPC C07C 2/76; B01J 29/7038; C10L 10/10; C10L 2270/023; C07C 2529/70; C10L 2200/0423; C10L 1/1608

(57) ABSTRACT

The present disclosure relates to processes for the alkylation of isoparaffins. A process may include introducing, in a multistage reactor, a solid acid catalyst to an isoparaffin feed and an olefin feed at a pressure of about 300 psig to about 1500 psig to form a alkylation product mixture. A process may also include solid acid catalyst that includes a crystalline microporous material of the MWW framework type. In yet other embodiments, the present disclosure provides for processes for the alkylation of an isoparaffin. A process may include introducing, in a multistage reactor, a solid acid catalyst to an isoparaffin feed and an olefin feed at a temperature of from about 100° C. to about 200° C. to form an alkylation product mixture. A process may further include a solid acid catalyst that includes a crystalline microporous material of the MWW framework type.

### CROSS-REFERENCE TO RELATED APPLICATIONS

This application claims the benefit of U.S. Provisional Application No. 62/883,267 filed Aug. 6, 2019, which is herein incorporated by reference in its entirety.

### FIELD OF THE INVENTION

The present disclosure relates to processes and apparatuses for alkylation of isoparaffins and, in particular, to processes and apparatuses for alkylation of isoparaffins with olefins to produce high octane rated additive for fuels, such as gasoline.

### BACKGROUND OF THE INVENTION

The alkylation of isoparaffins, such as isobutane, is an important refinery process for the production of high octane alkylate as a blend component for gasoline. Alkylation involves the addition of an alkyl group to an organic molecule. Thus, an isoparaffin can be reacted with an olefin to provide an isoparaffin of higher molecular weight. The product is a valuable blending component for gasoline due to its high octane rating, low sulfur, low olefin, and low aromatic content. Industrially, alkylation often involves the reaction of C2-05 olefins with, for example, isobutane in the presence of an acidic catalyst to form alkylates. Alkylates are valuable blending components for the manufacture of premium gasolines due to their high octane ratings.

In the past, alkylation processes have included the use of liquid acids, such as hydrofluoric acid or sulfuric acid as catalysts. The use of liquid acids provides challenges in disposal of spent acid streams. Furthermore, consideration has been given by regulatory authorities to the restriction of the use of liquid acids in industrial alkylation reactions. An alternative to liquid acids are solid acids, such as zeolites. However, some solid acids, such as faujasite, typically have short catalyst lifetimes which lead to frequent catalyst regeneration and increased costs and may further require the use of precious metals such as platinum and palladium in catalyst regeneration.

Recent efforts in further improving alkylation catalysts over liquid acid catalysts and previous solid acid catalysts have been focused on the development and use of solid acid catalysts, including zeolites, such as zeolites having the MWW framework type, e.g. MCM-22, MCM-36 and MCM-49 for the catalytic alkylation of an olefin with an isoparaffin. (U.S. Pat. Nos. 4,992,615, 5,254,792, 5,304,698, 5,354,718, 5,516,962). The previous approaches in alkylation of isoparaffins focused on using a single stage reactor where the feed isobutane to olefin ratio (i:o ratio), a volume to volume ratio, was set by the composition of the gas entering the single stage reactor. For liquid acids the i:o ratio has typically been 4:1 to 10:1, and for solid catalysts the i:o ratio has typically been 40:1 to 50:1, both based solely on the composition of the feedstock entering a single stage reactor.

The use of a single stage reactor may limit the ability to convert olefins and isoparaffins into higher octane rated fuel additives. For example, a single stage reactor does not permit splitting of the olefin feedstock creating a lower local concentration of olefin and a greater i:o ratio.

There remains a need for an improved isoparaffin-olefin alkylation processes that can be catalyzed by a solid acid catalyst with high conversion and high activity that maintains product quality of existing liquid phase processes.

### SUMMARY OF THE INVENTION

The present disclosure relates to processes for the alkylation of isoparaffins.

In an embodiment, a process may include introducing, in a multistage reactor, a solid acid catalyst to an isoparaffin feed and an olefin feed at a pressure of about 300 psig to about 1500 psig to form a alkylation product mixture. The process may also include solid acid catalyst that includes a crystalline microporous material of the MWW framework type.

In an embodiment, the present disclosure provides for processes for the alkylation of an isoparaffin. The process may include introducing, in a multistage reactor, a solid acid catalyst to an isoparaffin feed and an olefin feed at a temperature of from about 100° C. to about 200° C. to form an alkylation product mixture. The process may further include a solid acid catalyst that includes a crystalline microporous material of the MWW framework type.

### BRIEF DESCRIPTION OF THE DRAWING

FIG. 1A is a depiction of a reactor with one stage configured to receive an olefin feed and an isoparaffin feed.

FIG. 1B is a depiction of a reactor with two stages configured to receive an olefin feed and an isoparaffin feed, according to an embodiment.

FIG. 1C is a depiction of a reactor with four stages configured to receive an olefin feed and an isoparaffin feed, according to an embodiment.

FIG. 1D is a depiction of a reactor with eight stages configured to receive an olefin feed and an isoparaffin feed, according to an embodiment.

### DETAILED DESCRIPTION OF THE INVENTION

Previous alkylation processes and systems described in previous patent applications relied on a single stage reactor. Single stage alkylation reactors may provide lower conversion of isoparaffins and olefins into higher octane rated fuel additives, increased by-product formation, and can be limited in flow rate or i:o ratio.

It was believed that the addition of multiple stages would improve conversion because olefin interactions with active catalyst site could increase with additional stages including catalyst. Additionally, the use of multiple stages may allow for use of similar feeds, but provide a larger i:o ratio at each stage than a single stage reactor could provide. A multiple stage reactor may offer improvements over single stage processes including splitting of olefin introduction into various stages which decreases the local concentration of olefin in a catalyst bed, which may provide improved i:o ratios, decreased issues with catalyst deactivation, decreased byproduct formation, and improved conversion of isoparaffins. Improved conversion may result from increased olefin interactions with active catalyst sites resulting from passing over catalyst beds within additional reactor stages.

However, it has been discovered that performing the alkylation in multiple stages shows a decrease in conversion and increased loss of catalyst activity at each subsequent stage. Without being limited by theory, it is believed that olefin oligomerization creates higher olefins that block catalyst active sites decreasing alkylation of paraffins and reducing catalyst activity. Furthermore, the feed entering a later stage does not have the same chemical composition as the feed entering previous stages because the feed after the first stage contains some quantity of higher olefins. Additionally, the problem of higher olefin content may be multiplicative because a decrease in catalytic activity lowers isoparaffin conversion rates. At lower isoparaffin conversion rates more olefin oligomers are formed and, therefore, catalyst activity may decrease more quickly.

The benefits of a multistage reactor can be realized by avoiding production of olefin oligomers and/or removing the olefin oligomers if produced.

It has been discovered that high conversion rates, maintenance of high catalyst activity, and decreased production of olefin oligomers can be achieved by increasing the i:o ratio, increasing the reactor pressure, and/or increasing the reactor temperature. Increasing the reactor pressure or the reactor temperature may decrease the higher olefins produced and therefore decrease catalyst deactivation and increase conversion rates.

### Definitions

The term “Cn” compound (olefin or paraffin) where n is a positive integer, e.g., 1, 2, 3, 4, 5, etc., means a compound having n number of carbon atom(s) per molecule. The term “Cn+” compound where n is a positive integer, e.g., 1, 2, 3, 4, 5, etc., means a compound having at least n number of carbon atom(s) per molecule. The term “Cn−” compound where n is a positive integer, e.g., 1, 2, 3, 4, 5, etc., means a compound having no more than n number of carbon atom(s) per molecule.

The term “critical point” is the liquid-vapor end point of a phase equilibrium curve that designates conditions under which a liquid and vapor may coexist. At temperatures higher than the critical point (a “critical temperature”) a gas cannot be liquefied by pressure alone. At temperatures and pressures higher than the critical point the material is a supercritical fluid. For the purposes of this disclosure the critical point for isobutane is 134.6° C. and 3650 kPa, and the critical point for isopentane is 187.2° C. and 3378 kPa.

### Reactor Design and Conditions

The processes described can be conducted in any suitable multistage reactor, such as one including fixed-beds, moving beds, swing beds, fluidized beds (including turbulent beds), and/or one or more combinations thereof. A reactor stage begins at the point in which olefin is introduced and ends at either an interstage space or where additional olefin is introduced. A multistage reactor may have one or more interstage spaces between stages. An interstage space may be an open space, a filled space, a separation barrier, a distribution plate or system, or an injection point. Multistage reactors of the present disclosure may be configured to receive an olefin feed at multiple sites or inlets, and the introduction of olefin marks a new reactor stage. In addition, the reactor may include multiple catalyst beds located in the same or different housing. A multistage reactor or a stage within the reactor may include a bed of catalyst particles where the particles have insignificant motion in relation to the bed (a fixed bed). In addition, injection of the olefin feed can be effected at a single point in the reactor or at multiple points spaced along the reactor. The isoparaffin feed and the olefin feed may be premixed before entering the reactor.

In certain embodiments of the present disclosure, the multistage reactor includes a plurality of fixed beds, continuous flow-type reactor stages in either a down flow or up flow mode, where the reactor stages may be arranged in series or parallel. The multistage reactor may include multiple reactor stages in series and/or in parallel, for example, a multistage reactor may include 2 stages, 4 stages, 8 stages, 10 stages, 12 stages, or any other plurality of stages. A reactor stage includes a catalyst bed. The reactor stage may have various configurations such as: multiple horizontal beds, multiple parallel packed tubes, multiple beds each in its own reactor shell, or multiple beds within a single reactor shell. In certain embodiments, a reactor stage includes a fixed bed which provides uniform flow distribution over the entire width and length of the bed to utilize substantially all of the catalyst. In at least one embodiment, the multistage reactor can provide heat transfer from reactor stages or catalyst beds in order to provide effective methods for controlling temperature.

The efficiency of a multistage reactor containing fixed beds of catalyst may be affected by the pressure drop across a fixed bed. The pressure drop depends on various factors such as the path length, the catalyst particle size, and pore size. A pressure drop that is too large may cause channeling through the catalyst bed, poor efficiency, and increased catalyst deactivation. In some embodiments, the reactor has a cylindrical geometry with axial flows through the catalyst beds. The various designs of the multistage reactor may accommodate control of specific process conditions, e.g. pressure, temperature, LHSV, and OLHSV (olefin liquid hourly space velocity). The combination of LHSV and OLHSV determine catalyst volume and residence time that may provide the desired conversion.

Operating pressures may be controlled to reduce or eliminate oligomerization reactions and/or favor alkylation reactions. Additionally, increased reactor pressures may improve conversion rates for the olefin feed and improve selectivity towards the alkylated paraffin over olefin oligomers. Operating pressure may be from about 300 to about 1500 psig (about 2068 to about 10342 kPag), such as from about 400 to about 1200 psig (about 2758 to about 8274 kPag), from about 450 psig to about 1000 psig (about 3102 to about 6895 kPag), from about 550 psig to about 950 psig (about 3792 to about 6550 kPag), from about 650 psig to about 950 psig (about 4481 to about 6550 kPag), from about 750 psig to about 950 psig (about 5171 to about 6550 kPag), or from about 800 psig to about 950 psig (about 5516 to about 6550 kPag). In some embodiments, the operating temperature and pressure remain above the critical point for the isoparaffin feed during the reactor run.

Additionally, operating temperatures may be controlled to reduce or eliminate olefin oligomerization reactions and/or favor alkylation of isoparaffins. Operating temperature may be from about 100° C. or greater, such as about 130° C. or greater, about 140° C. or greater, about 150° C. or greater, or about 160° C. or greater, such as from about 100° C. to about 200° C., from about 130° C. to about 170° C., or from about 140° C. to about 160° C. Operating temperatures may exceed the critical temperature of the isoparaffin feed, or the principal component in the isoparaffin feed. The term “principal component” is defined as the component of highest concentration in the feedstock. For example, isobutane is the principal component in a feedstock consisting of isobutane and 2-methylbutane in isobutane: 2-methylbutane weight ratio of about 50:1.

The temperature of the multistage reactor or an individual stage within the reactor may affect by-product formation and a temperature higher than 130° C. may decrease heavier olefin concentrations. Furthermore, an increase in temperature may improve conversion of the olefin feed. However, for certain olefins, a higher temperature increases olefin isomerization, and olefin isomerization may lead to the formation of alkylation products that are lower in value. For example, in the alkylation of isobutane with 2-butene, a main component of the alkylation product mixture is trimethylpentane which has an octane rating of 100, but if 2-butene is isomerized to 1-butene the alkylation shifts to higher production of dimethylhexane which has an octane rating of 70, providing less value as a fuel additive. Therefore, temperature may be used to reduce or eliminate heavier olefin concentrations, especially in cases where the olefin is not affected by isomerization, such as propene or isobutene. In some embodiments, the alkylation product mixture contains about 10 wt % or less, such as about 5 wt % or less, about 2 wt % or less, about 1 wt % or less, or is substantially free of products of olefin oligomerization.

Hydrocarbon flow through a reactor stage containing the catalyst is typically controlled to provide an olefin liquid hourly space velocity (OLHSV) sufficient to convert about 99 percent, or more, by weight of the fresh olefin to alkylation product. In some embodiments, OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The liquid hourly space velocity of the isoparaffin is controlled to meet a target i:o ratio. Because the i:o ratio is vol:vol, the isoparaffin liquid hourly space velocity is directly correlated to the OLHSV.

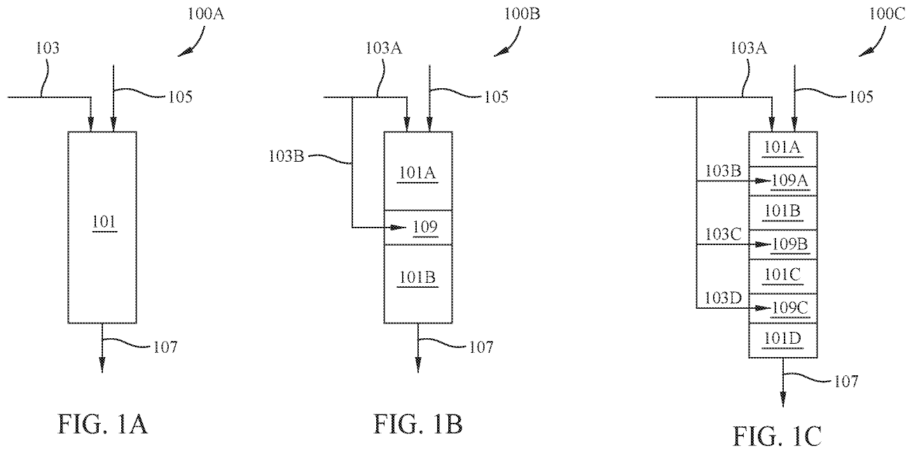

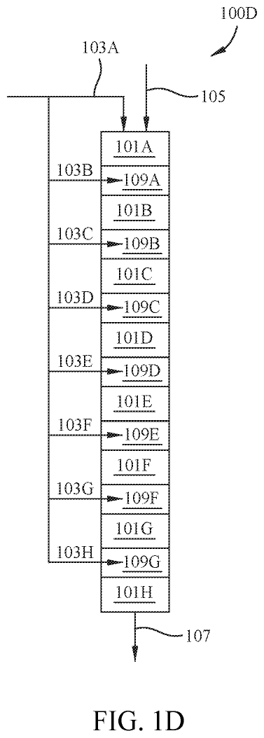

FIG. 1A depicts an alkylation reactor 100A with a single reactor stage 101. Reactor stages(s) may individually or collectively be termed an alkylation zone and include catalyst, such as a solid acid catalyst including zeolite of the MWW framework type. The olefin feed is introduced to reactor stage 101 via line 103 and the isoparaffin feed through line 105. An alkylation product mixture exits the reactor through line 107. In alkylation reactor 100A the i:o ratio is controlled solely by the composition of the olefin feed and the isoparaffin feed entering reactor bed 101.

FIG. 1B depicts a multistage alkylation reactor 100B with two reactor stages: first stage 101A and second stage 101B. The olefin feed is introduced to the reactor beds via lines 103A and 103B and OLHSV values are from about 0.01 hr⁻¹ to about 10 hr 1, such as about 0.02 hr⁻¹ to about 1 hr⁻¹, or such as about 0.03 hr⁻¹ to about 0.1 hr⁻¹. The split introduction of the olefin feed allows a lower concentration (half) of the olefin feed to be introduced locally to each of the first stage 101A and the second stage 101B. The isoparaffin feed is introduced to alkylation reactor 100B through line 105. Alkylation reactor 100B has an interstage space 109 between first stage 101A and the second stage 101B to allow for introduction of additional olefin feed through line 103B. If lines 103 and 105 have the same composition as in FIG. 1A, then the local i:o ratio is doubled in the configuration of FIG. 1B because the olefin feed is divided into two lines 103A and 103B and the olefin introduced via line 103A to first stage 101A can be converted, such as about 90 wt % or greater, 95 wt % or greater, 98 wt % or greater, or 99 wt % or greater is converted in the reaction within first stage 101A, based on the total weight of olefin in the olefin feed introduced via line 103A. Additionally, only a small portion of the isoparaffin feed is converted by the reaction in first stage 101A, such as about 10 wt % or less, 5 wt % or less, 2 wt % or less, 1 wt % or less, 0.5 wt % or less, or 0.1 wt % or less, of the isoparaffin feed is converted based on the total weight of isoparaffin. Therefore, the amount of isoparaffin introduced to interstage 109 and, therefore, introduced to second stage 101B is similar to that introduced to first stage 101A. For example, if an i:o ratio of 100:1 is introduced to first stage 101A and there is an olefin conversion of 100% then the i:o ratio in second stage 101B would be ˜99:1, if no additional isoparaffin was added. Furthermore, the olefin introduced to interstage 109 (either via line 103B or from the effluent of first stage 101A) and, therefore, introduced to second stage 101B is similar in quantity to that introduced to first stage 101A. Additionally, because the selected isoparaffin may be consumed in each stage, such as in amounts of about 10 wt % or less, about 5 wt % or less, about 2 wt % or less, about 1 wt % or less, about 0.5 wt % or less, or about 0.1 wt % or less, additional isoparaffin may be added in an interstage space so as to maintain a consistent i:o ratio throughout the multistage reactor. Similarly to FIG. 1A, an alkylation product mixture exits the reactor through line 107.

FIG. 1C depicts a multistage alkylation reactor 100C with four reactor stages: first stage 101A, second stage 101B, third stage 101C, and fourth stage 101D. The olefin feed is introduced to the reactor beds via lines 103A, 103B, 103C and 103D and OLHSV values are from about 0.01 hr⁻¹ to about 10 hr⁻¹, such as about 0.02 hr⁻¹ to about 1 hr 1, or such as about 0.03 hr¹ to about 0.1 hr⁻¹. The split introduction of the olefin feed allows a lower concentration (one quarter) of the olefin feed to be introduced locally to each of the first stage 101A, second stage 101B, third stage 101C, and fourth stage 101D. The isoparaffin feed is introduced to alkylation reactor 100C through line 105. Alkylation reactor 100C has multiple interstage spaces: first interstage space 109A, second interstage space 109B, and third interstage space 109C between reactor stages 101A, 101B, 101C, and 101D to allow for introduction of additional olefin feed through lines 103B, 103C, and 103D. If lines 103 and 105 have the same composition as in FIG. 1A, then the local i:o ratio is 4 times that found in FIG. 1A, because the olefin feed is divided into four lines 103A, 103B, 103C, and 103D. The i:o ratio in a single stage is only slightly affected by prior stage(s) because the olefin introduced to a prior stage can be largely converted within that stage, but the isoparaffin is introduced at such a ratio that the amount converted may have little effect on the ratio in later stages. For example, the olefin introduced via line 103A to first stage 101A is converted, such as about 90 wt % or greater, 95 wt % or greater, 98 wt % or greater, or 99 wt % or greater is converted in first stage 101A, based on the total weight of olefin in the olefin feed introduced via line 103A. Additionally, only a small portion of the isoparaffin feed may be converted by the reaction in first stage 101A, such as about 10 wt % or less of the isoparaffin feed is converted based on the total weight of isoparaffin introduced to first stage 101A, such as 5 wt % or less, 2 wt % or less, 1 wt % or less, 0.5 wt % or less, or 0.1 wt % or less. Therefore, the amount of isoparaffin introduced to interstage 109A and, therefore, introduced to second stage 101B is similar to that introduced to first stage 101A and the olefin introduced to interstage space 109A via line 103B and from the effluent of first stage 101A serves to bring the olefin level back up to a desired i:o ratio. For example, if an i:o ratio of 100:1 is introduced to first stage 101A and there is an olefin conversion of 100%, then the i:o ratio in second stage 101B would be ˜99:1, if no additional isoparaffin was added. The combination of isoparaffin and olefin is then introduced to second stage 101B, where the olefin may be converted in second stage 101B, such as about 90 wt % or greater, 95 wt % or greater, 98 wt % or greater, or 99 wt % or greater is converted in second stage 101B, based on the total weight of olefin introduced to interstage space 109A. Similarly, only a small portion of the isoparaffin feed may be converted by the reaction in second stage 101B, such as about 10 wt % or less of the isoparaffin feed is converted based on the total weight of isoparaffin introduced to interstage space 109A, such as 5 wt % or less, 2 wt % or less, 1 wt % or less, 0.5 wt % or less, or 0.1 wt % or less. As the isoparaffin feed enters additional interstage spaces (such as second interstage space 109B, and third interstage space 109C) more olefin may be introduced (via lines 103C and 103D) to adjust the i:o ratio as the combined feeds are introduced to additional stages (such as third stage 101C and fourth stage 101D). Additionally, because the selected isoparaffin may be consumed in each stage (such as in amounts of about 10 wt % or less, about 5 wt % or less, about 2 wt % or less, about 1 wt % or less, about 0.5 wt % or less, or about 0.1 wt % or less), additional isoparaffin may be added in an interstage space so as to maintain a consistent i:o ratio throughout the multistage reactor. Similarly to FIG. 1A, an alkylation product mixture exits the reactor through line 107.

FIG. 1D depicts a multistage alkylation reactor 100D with eight reactor stages: first stage 101A, second stage 101B, third stage 101C, fourth stage 101D. The olefin feed is introduced to the reactor beds via lines 103A, 103B, 103C and 103D and OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The split introduction of the olefin feed allows a lower concentration (one quarter) of the olefin feed to be introduced locally to each of the first stage 101A, second stage 101B, third stage 101C, fourth stage 101D, fifth stage 101E, sixth stage 101F, seventh stage 101G, and eighth stage 101H. The isoparaffin feed is introduced to alkylation reactor 100D through line 105. Alkylation reactor 100D has multiple interstage spaces: first interstage space 109A, second interstage space 109B, third interstage space 109C, fourth interstage space 109D, fifth interstage space 109E, sixth interstage space 109F, and seventh interstage space 109G between reactor stages 101A, 101B, 101C, 101D, 101E, 101F, 101G, and 101H to allow for introduction of additional olefin feed through lines 103B, 103C, 103D, 103E, 103F, 103G, and 103H. If lines 103 and 105 have the same composition as in FIG. 1A, then the local i:o ratio is 8 times that found in FIG. 1A, because the olefin feed is divided into eight lines 103A, 103B, 103C, 103D, 103E, 103F, 103G, and 103H. The i:o ratio in a single stage is only slightly affected by prior stage(s) because the olefin introduced to a prior stage can be largely converted within that stage, but the isoparaffin is introduced at such a ratio that the amount converted may have little effect on the ratio in later stages. For example, the olefin introduced via line 103A to first stage 101A is converted, such as about 90 wt % or greater, about 95 wt % or greater, about 98 wt % or greater, or about 99 wt % or greater is converted in first stage 101A, based on the total weight of olefin in the olefin feed introduced via line 103A. Additionally, only a small portion of the isoparaffin feed may be converted by the reaction in first stage 101A, such as about 10 wt % or less of the isoparaffin feed is converted based on the total weight of isoparaffin introduced to first stage 101A, such as about 5 wt % or less, about 2 wt % or less, about 1 wt % or less, about 0.5 wt % or less, or about 0.1 wt % or less. Therefore, the amount of isoparaffin introduced to interstage 109A and, therefore, introduced to second stage 101B is similar or slightly less than that introduced to first stage 101A and the olefin introduced to interstage space 109A via line 103B and from the effluent of first stage 101A serves to bring the olefin level back up to a desired i:o ratio. Therefore, for example, if an i:o ratio of 100:1 is introduced to first stage 101A and there is an olefin conversion of 100% then the i:o ratio in second stage 101B would be ˜99:1, if no additional isoparaffin was added. The combination of isoparaffin and olefin is then introduced to second stage 101B, where the olefin may be converted in second stage 101B, such as about 90 wt % or greater, about 95 wt % or greater, about 98 wt % or greater, or about 99 wt % or greater is converted in second stage 101B, based on the total weight of olefin introduced to interstage space 109A. Similarly, only a small portion of the isoparaffin feed may be converted by the reaction in second stage 101B, such as about 10 wt % or less of the isoparaffin feed is converted based on the total weight of isoparaffin introduced to interstage space 109A, such as about 5 wt % or less, about 2 wt % or less, about 1 wt % or less, about 0.5 wt % or less, or about 0.1 wt % or less. As the isoparaffin feed enters additional interstage spaces (such as second interstage space 109B, and third interstage space 109C) more olefin may be introduced (via lines 103C, 103D, 103E, 103F, 103G, and 103H) to adjust the i:o ratio as the combined feeds are introduced to additional stages (such as third stage 101C, fourth interstage space 109D, fifth interstage space 109E, sixth interstage space 109F, and seventh interstage space 109G). Additionally, because the selected isoparaffin may be consumed in each stage, such as in amounts of about 10 wt % or less, about 5 wt % or less, about 2 wt % or less, about 1 wt % or less, about 0.5 wt % or less, or about 0.1 wt % or less, additional isoparaffin may be added in an interstage space so as to maintain a consistent i:o ratio throughout the multistage reactor. Similarly to FIG. 1A, an alkylation product mixture exits the reactor through line 107.

### Feedstocks

Feedstocks useful in the present alkylation process include at least one isoparaffin feed and at least one olefin feed. The isoparaffin feed used in alkylation processes of the present disclosure may have from about 4 to about 7 carbon atoms. Representative examples of such isoparaffins include isobutane, isopentane, 3-methylhexane, 2-methylhexane, 2,3-dimethylbutane, and mixture(s) thereof, typically isobutane.

The olefin component of the feedstock may include at least one olefin having from 2 to 12 carbon atoms. Representative examples of such olefins include ethylene, propylene, 1-butene, 2-butene, isobutylene, 1-pentene, 2-pentene, 3-pentene, 2-methyl-1-butene, 3-methyl-1-butene, 2-methyl-2-butene, hexene, octene, heptene, or mixture(s) thereof. In some embodiments, the olefin component of the feedstock is selected from the group consisting of propene, 1-butene, 2-butene, isobutylene, 1-pentene, 2-pentene, 3-pentene, 2-methyl-1-butene, 3-methyl-1-butene, 2-methyl-2-butene, and mixture(s) thereof. For example, in one embodiment, the olefin component of the feedstock may include a mixture of propylene and at least one butene, such as 2-butene, where the weight ratio of propylene to butene is from about 0.01:1 to about 1.5:1, such as from about 0.1:1 to about 1:1. In another embodiment, the olefin component of the feedstock may include a mixture of propylene and at least one pentene, where the weight ratio of propylene to pentene is from about 0.01:1 to about 1.5:1, such as from about 0.1:1 to about 1:1.

The concentration of olefin feed can be adjusted by, e.g., staged additions thereof. By staged additions, isoparaffin/olefin feed concentrations (and therefore the i:o ratio) can be maintained at levels to improve conversion and reduce catalyst deactivation. In at least one embodiment, the ratio of isoparaffin to olefin ratio by volume, referred to as the i:o ratio is: about 100:1 or greater, about 120:1 or greater, about 140:1 or greater, about 160:1 or greater, about 180:1 or greater, about 200:1 or greater, about 220:1 or greater, about 240:1 or greater, about 260:1 or greater, about 280:1 or greater, or about 300:1 or greater, such as from about 100:1 to about 500:1, about 120:1 to about 500:1, about 160:1 to about 480:1, about 200:1 to about 450:1, about 220:1 to about 450:1, about 240:1 to about 420:1, or about 240:1 to about 400:1.

The production of olefin oligomers increases with lower i:o ratios. To reduce or eliminate the production of olefin oligomers an i:o ratio of about 100:1 or greater may be used. On the other hand, the efficiency of the alkylation process can be reduced at higher i:o ratios, due to large quantity of isoparaffin present in the alkylation product mixture, which is then separated and recycled to the reactor. The separation and recycling of isoparaffin may occur in a distillation apparatus that allows for distillation of low C5-alkane from C6+ alkanes and alkenes produced in the reactor. A higher i:o ratio can provide greater quantities of C5-alkane separated from the alkylation product mixture that can be recycled to the reactor.

Before being sent to the reactor, the isoparaffin feed and/or olefin feed may be treated to remove catalyst poisons. For example, catalyst poisons may be removed using guard beds with specific absorbents for reducing the level of S, N, and/or oxygenates to values which do not affect catalyst stability, activity, and selectivity.

### Catalyst

Catalysts suitable for use in the systems and processes described are crystalline microporous materials of the MWW framework type. The term “crystalline microporous material of the MWW framework type” and grammatical variants thereof includes one or more of:

(a) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);

(b) molecular sieves made from a common second degree building block, being a 2-dimensional tiling of such MWW framework topology unit cells, forming a monolayer of one unit cell thickness, such as one c-unit cell thickness;

(c) molecular sieves made from common second degree building blocks, being layers of one or more than one unit cell thickness, where the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and

(d) molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084; EMM-10 (described in U.S. Pat. No. 7,842,277), EMM-12 (described in U.S. Pat. No. 8,704,025), EMM-13 (described in U.S. Pat. No. 8,704,023), MIT-1 (Luo, et. al., Chem Sci. 2015 Nov. 1; 6(11): 6320-6324), and mixture(s) thereof.

In some embodiments, the crystalline microporous material of the MWW framework type employed may be an aluminosilicate material having a silica to alumina molar ratio of about 10 or more, such as from about 10 to about 50.

In some embodiments, the crystalline microporous material of the MWW framework type employed may be contaminated with other crystalline materials, such as ferrierite or quartz. These contaminants may be present in quantities about 10 wt % or less, such as about 5 wt % or less.

### Binder

Catalysts suitable for use in the systems and processes described include a binder.

Binder materials, including other inorganic oxides than alumina, such as silica, titania, zirconia and mixtures and compounds thereof, may be present in the catalyst in amounts about 90 wt % or less, for example about 80 wt % or less, such as about 70 wt % or less, for example about 60 wt % or less, such as about 50 wt % or less. Where a non-alumina binder is present, the amount employed may be as little as about 1 wt %, such as about 5 wt % or more, for example about 10 wt % or more. In at least one embodiment, a silica binder is employed such as disclosed in U.S. Pat. No. 5,053,374, incorporated by reference. In other embodiments, a zirconia or titania binder is used.

In other embodiments, the binder may be a crystalline oxide material such as the zeolite-bound-zeolites described in U.S. Pat. Nos. 5,665,325 and 5,993,642, incorporated by reference. In the case of crystalline binders, the binder material may contain alumina, including amorphous alumina.

### Product

The product of the alkylation reaction (also referred to as the alkylation product mixture) can include: alkanes resulting from the alkylation of isoparaffin with olefin, unreacted isoparaffin, unreacted olefin, olefin oligomers, other byproducts, including other alkanes and alkenes. The product composition of the isoparaffin-olefin alkylation reaction described is dependent on the reaction conditions and the composition of the olefin feed and isoparaffin feed. The product is a complex mixture of hydrocarbons, since alkylation of the feed isoparaffin by the feed olefin is accompanied by a variety of competing reactions including cracking, olefin oligomerization, and/or further alkylation of the alkylate product by the feed olefin. For example, in the case of alkylation of isobutane with C3-05 olefins, such as 2-butene, the product may include about 20-30 wt % of C5-C7 hydrocarbons, 50-75 wt % of C8 hydrocarbons and 2.5-20 wt % of C9+ hydrocarbons. Moreover, using an MWW type molecular sieve as the catalyst, it has been discovered that processes can be selective to desirable high octane components so that, in the case of alkylation of isobutane with C3-05 olefins, the C6 fraction typically includes at least 40 wt %, such as at least 70 wt %, of 2,3-dimethylbutane, the C7 fraction typically includes at least 40 wt %, such as at least 80 wt %, of 2,3-dimethylpentane and the C8 fraction typically includes at least 50 wt %, such as at least 70 wt %, of 2,3,4-trimethylpentane; 2,3,3-trimethylpentane; and 2,2,4-trimethylpentane.

Additionally, in the case of alkylation of isobutane with C5 olefins, such as n-pentene and 2-methyl-2-butene, the product may include about 30-40 wt % of C5 hydrocarbons, 15-25 wt % of C9 hydrocarbons, 25-35 wt % of C8 hydrocarbons, and 2.5-10 wt % of C10+ hydrocarbons. Moreover, using an MWW type molecular sieve as the catalyst, it has been found that a process can be selective to desirable high octane components so that, in the case of alkylation of isobutane with C5 olefins, the C8 and C9 fractions typically include a higher molar ratio of trimethyl isomers to dimethyl isomers, which is beneficial for increasing octane. For the C8 fraction, the molar ratio of trimethylpentane to dimethylhexane can be about 3 or more, e.g. about 4 to about 5, or about 3 to about 6. For the C9 fraction, the molar ratio of trimethylhexane to dimethylheptane can be about 1 or more, e.g. about 1.5 or more, or from about 1 to about 3.

The product of the isoparaffin-olefin alkylation reaction may be fed to a separation system, such as a distillation train, to recover a C5+ fraction for use as a gasoline octane enhancer. Additionally, the separation system may separate the C4-C6 isoparaffin to be recycled as part or all of the isoparaffin feed. Furthermore, depending on alkylate demand, part or all of a C9+ fraction can be recovered for use as a distillate blending stock.

### Embodiments of the Present Disclosure

Clause 1. A process for the alkylation of an isoparaffin, the process including:

introducing, in a multistage reactor, a solid acid catalyst to an isoparaffin feed and an olefin feed at a pressure of about 300 psig to about 1500 psig to form a alkylation product mixture;

where the solid acid catalyst includes a crystalline microporous material of the MWW framework type.

Clause 2. The process of clause 1, where the pressure is about 450 psig or greater.

Clause 3. The process of clause 1, where the pressure is about 750 psig or greater.

Clause 4. The process of any of clauses 1 to 3, where the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or mixture(s) thereof.

Clause 5. The process of any of clauses 1 to 4, where the isoparaffin feed includes isobutane.

Clause 6. The process of clause 5, where the contacting a solid acid catalyst with an isoparaffin feed, and an olefin feed takes place at a pressure and a temperature greater than the critical point of isobutane.

Clause 7. The process of any of clauses 1 to 4, where the isoparaffin feed includes isopentane.

Clause 8. The process of clause 7, where the contacting a solid acid catalyst with an isoparaffin feed, and an olefin feed is performed at a pressure and a temperature greater than the critical point of isopentane.

Clause 9. The process of any of clauses 1 to 8, where the olefin feed includes one or more C2-05 olefins.

Clause 10. The process of any of clauses 1 to 9, wherein the olefin feed includes propene, 1-butene, 2-butene, isobutylene, 1-pentene, 2-pentene, 3-pentene, 2-methyl-1-butene, 3-methyl-1-butene, 2-methyl-2-butene, or a combination thereof.

Clause 11. The process of any of clauses 1 to 10, where the olefin feed includes propene, 1-butene, 2-butene, isobutylene, or a combination thereof.

Clause 12. The process of any of clauses 1 to 11, where the alkylation product mixture includes less than 5 wt % C8+ olefins.

Clause 13. The process of any of clauses 1 to 12, where introducing the solid acid catalyst to an isoparaffin feed and an olefin feed is performed at a ratio of isoparaffin:olefin of about 120:1 or greater.

Clause 14. A process for the alkylation of an isoparaffin, the process including:

introducing, in a multistage reactor, a solid acid catalyst to an isoparaffin feed and an olefin feed at a temperature of from about 100° C. to about 200° C. to form an alkylation product mixture,

where the solid acid catalyst includes a crystalline microporous material of the MWW framework type.

Clause 15. The process of clause 14, where the temperature is about 130° C. or greater.

Clause 16. The process of clause 14, where the temperature is about 140° C. or greater.

Clause 17. The process of any of clauses 14 to 16, where the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or mixtures of two or more thereof.

Clause 18. The process of any of clauses 14 to 17, where the isoparaffin feed includes isobutane.

Clause 19. The process of clause 18, where contacting the solid acid catalyst with an isoparaffin feed and an olefin feed is performed at a pressure and a temperature greater than the critical point of isobutane.

Clause 20. The process of any of clauses 14 to 17, where the isoparaffin feed includes isopentane.

Clause 21. The process of clause 20, where contacting the solid acid catalyst with an isoparaffin feed and an olefin feed is performed at a pressure and a temperature greater than the critical point of isopentane.

Clause 22. The process of any of clauses 14 to 21, where the olefin feed includes one or more C2-05 olefins.

Clause 23. The process of any of clauses 14 to 21, where the olefin feed includes one or more of propene, 1-butene, or 2-butene.

Clause 24. The process of any of clauses 14 to 23, where the alkylation product mixture includes less than 5 wt % C8+ olefins.

Clause 25. The process of any of clauses 14 to 24, where introducing the solid acid catalyst to an isoparaffin feed and an olefin feed is performed at a ratio of isoparaffin:olefin of about 120:1 or greater.

### EXAMPLES

### Feed Pretreatment

Isobutane was obtained from a commercial source and used as received. The isobutene purity was 99.6% with the balance n-butane.

Propylene and 2-butene were obtained from a commercial specialty gases source and used as received. The 2-butene was a mixture of trans-2-butene and cis-2-butene.

### Catalyst Preparation and Loading

Catalysts used for isobutane alkylation with light olefins are dried in the reactor under nitrogen flow at 250° C. for at least 4 hours prior to use.

### Example 1

The catalyst was prepared by combining 80 parts MCM-49 zeolite crystals with 20 parts pseudoboehmite alumina, on a calcined dry weight basis. The MCM-49 and pseudoboehmite alumina dry powder were placed in a muller or a mixer and mixed for 30 minutes. Sufficient water was added to the MCM-49 and alumina during the mixing process to produce an extrudable paste. The extrudable paste was formed into a 1/20 inch quadralobe extrudate using an extruder. After extrusion, the extrudate was dried at a temperature ranging from 250° F. (121° C.) to 325° F. (168° C.). After drying, the dried extrudate was heated to 1000° F. (538° C.) under flowing nitrogen. The extrudate was then cooled to ambient temperature, humidified with saturated air or steam and then ion exchanged with 0.75 N ammonium nitrate solution followed by washing with deionized water and drying. The extrudate was then calcined in a nitrogen/air mixture to a temperature of 1000° F. (538° C.).

### Example 2

95 parts MCM-49 zeolite crystals were combined with 5 parts pseudoboehmite alumina, on a calcined dry weight basis. The MCM-49 and pseudoboehmite alumina dry powder were placed in a muller or a mixer and mixed for 30 minutes. Sufficient water was added to the MCM-49 and alumina during the mixing process to produce an extrudable paste. The extrudable paste was formed into a 1/20 inch quadralobe extrudate using an extruder. After extrusion, the extrudate was dried at a temperature ranging from 250° F. (121° C.) to 325° F. (168° C.). After drying, the dried extrudate was heated to 1000° F. (538° C.) under flowing nitrogen. The extrudate was then cooled to ambient temperature, humidified with saturated air or steam and then ion exchanged with 0.75 N ammonium nitrate solution followed by washing with deionized water and drying. The extrudate was then calcined in a nitrogen/air mixture to a temperature of 1000° F. (538° C.).

### Example 3

95 parts MCM-49 zeolite crystals were combined with 5 parts pseudoboehmite alumina, on a calcined dry weight basis. The MCM-49 and pseudoboehmite alumina dry powder were placed in a muller or a mixer and mixed for 30 minutes. Sufficient water was added to the MCM-49 and alumina during the mixing process to produce an extrudable paste. The temperature ranging from 250° F. (121° C.) to 325° F. (168° C.). After drying, the dried extrudate was ion exchanged with 0.75 N ammonium nitrate solution followed by washing with deionized water and drying. The dried extrudate was then heated to 1000° F. (538° C.) under flowing nitrogen and finally calcined in a nitrogen/air mixture to a temperature of 1000° F. (538° C.).

### Example 4

95 parts MCM-49 zeolite crystals were combined with 2.5 parts precipitated silica and 2.5 parts colloidal silica, on a calcined dry weight basis. The MCM-49 and precipitated silica dry powders were placed in a muller or a mixer and mixed for 20 minutes. Colloidal silica, available as Ludox HS-40 from W. R. Grace, was then added and mixed for about 5 to 10 minutes. Sufficient water and a 5% NaOH solution (2.5% NaOH by weight) were then added during the mixing process to produce an extrudable paste. The extrudable paste was formed into a 1/20 inch cylindrical extrudate using an extruder. After extrusion, the extrudate was dried at a temperature ranging from 250° F. (121° C.) to 325° F. (168° C.). After drying, the dried extrudate was ion exchanged with 0.75 N ammonium nitrate solution followed by washing with deionized water and drying. The dried extrudate was then heated to 1000° F. (538° C.) under flowing nitrogen and finally calcined in a nitrogen/air mixture to a temperature of 1000° F. (538° C.).

### Example 5

The catalyst of Example 1 was loaded into a pilot plant and operated as a single stage reactor, as shown in FIG. 1A. The reactor was 60″ long and made from ¾″ O.D. Schedule 40 pipe. The reactor was loaded with 50 g of catalyst. The reactor was located in an isothermal sand bath maintained at 302° F. (150° C.). Reactor pressure was 750 psig (5171 kPag). Isobutane (99.6% purity) and 2-butene were independently fed to the top of the single stage reactor at a relative rate such that the isobutane to 2-butene ratio at the top of the catalyst bed was 40:1. The reactor effluent was measured using a FID GC equipped with a 150 m Petrocol column. The 2-butene flow to the reactor was set to achieve an Olefin Liquid Hourly Space Velocity (OLHSV) of 0.06 h⁻¹ and subsequently 0.03 h⁻¹. Isobutane flowrates were adjusted as olefin flowrates were adjusted to maintain a constant i:o of 40:1 to the inlet to the catalyst bed. Average 2-butene conversion at 0.06 h⁻¹ was 80.7% and at 0.03 h⁻¹ the average 2-butene conversion was 93.7%.

### Example 6

The catalyst of Example 1 was loaded into a pilot plant and operated as a 2 stage reactor, as shown in FIG. 1B. Each stage was 60″ long and made from ¾″ O.D. Schedule 40 pipe. Each stage was loaded with 50 g of catalyst. The two-stage reactor was located in an isothermal sand bath maintained at 302° F. (150° C.). Reactor pressure was 750 psig (5171kPag). Isobutane (99.6% purity) was fed to the first stage of the reactor and the 2-butene flow was split evenly into 2 using Coriolis meters and independently fed to each stage. The relative rates of isobutane and 2-butene were set such that the isobutane to 2-butene ratio at the top of the first stage was 40:1. The alkylation product mixture exiting the reactor was measured using a FID GC equipped with a 150 m Petrocol column. The total 2-butene flow to the reactor was set to achieve an Olefin Liquid Hourly Space Velocity (OLHSV) of 0.06 h⁻¹. Average 2-butene conversion at 0.06 h⁻¹ was 70.4%. As can be seen when comparing the results to Example 5, the operation of the 2 stage system resulted in significantly lower olefin conversion.

### Example 7

The catalyst of Example 1 was loaded into a pilot plant and operated as a 4-stage reactor, as shown in FIG. 1C. Each stage was 60″ long and made from ¾″ O.D. Schedule 40 pipe. Each stage was loaded with 50 g of catalyst. The four-stage reactor was located in an isothermal sand bath maintained at 302° F. (150° C.). Reactor pressure was 750 psig (5171 kPag). Isobutane (99.6% purity) was fed to the first stage and the 2-butene flow was split evenly into 4 using Coriolis meters and independently fed to each stage of the four-stage reactor. The relative rates of isobutane and 2-butene were set such that the isobutane to 2-butene ratio at the top of the first stage was 40:1. The alkylation product mixture exiting the reactor was measured using a FID GC equipped with a 150 m Petrocol column. The total 2-butene flow to the reactor was set to achieve an Olefin Liquid Hourly Space Velocity (OLHSV) of 0.06 h⁻¹ and subsequently 0.03 h⁻¹. Isobutane flowrates were adjusted as olefin flowrates were adjusted to maintain a constant i:o of 40:1 to the inlet to the first stage. Average 2-butene conversion at 0.06 h⁻¹ was 61.4% and at 0.03 h⁻¹ the average 2-butene conversion was 77.5%. As can be seen when comparing the results to Example 5, the operation of the 4 bed system resulted in significantly lower olefin conversion.

### Example 8

The catalyst of Example 2 was loaded into a pilot plant and operated as a 4-stage reactor, as shown in FIG. 1C. Each stage was 60″ long and made from ¾″ O.D. Schedule 40 pipe. Each stage was loaded with 150 g of catalyst. The reactor was located in an isothermal sand bath maintained at 302° F. (150° C.). Reactor pressure was 750 psig (5171 kPag). Isobutane (99.6% purity) was fed to the first stage and the 2-butene flow was split evenly into 4 using Coriolis meters and independently fed to each reactor bed. The relative rates of isobutane and 2-butene were set such that the isobutane to 2-butene ratio at the top of the first stage was 40:1. The alkylation product mixture exiting the multistage reactor was measured using a FID GC equipped with a 150 m Petrocol column. The total 2-butene flow to the reactor was set to achieve an Olefin Liquid Hourly Space Velocity (OLHSV) of 0.03 h⁻¹. Average 2-butene conversion at 0.03 h⁻¹ was about 82%. The alkylation product mixture exiting the multistage reactor was sent to a distillation column for separation of C4 and lighter components from the reaction product. The alkylation product mixture was analyzed via offline GC and shown to have about 13.9% C5+ olefins.

### Example 9

A sample of the alkylation product mixture produced in Example 7 was hydrogenated using a commercial MaxSat™ hydrogenation catalyst available from ExxonMobil Catalyst & Licensing. Hydrogenation took place in a batch reactor at 200° C. and 800 psig (5171 kPag) for 8 hours. The hydrogenated alkylate was analyzed by GC and shown to have <1% C5+ olefins.

### Example 10

The catalyst of Example 2 was loaded into a pilot plant single stage reactor, as shown in FIG. 1A. The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor. The alkylation product mixture was analyzed using a FID GC equipped with a 150 m Petrocol column. The flow to the reactor was set to achieve an Olefin Liquid Hourly Space Velocity (OLHSV) of 0.057 h⁻¹. Average 2-butene conversion at 0.057 h⁻¹ was 99.7% Example 11

The catalyst of Example 2 was loaded into a pilot plant single stage reactor, as shown in FIG. 1A. The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor. The alkylation product mixture was analyzed using a FID GC equipped with a 150 m Petrocol column. The flow to the reactor was set to achieve an Olefin Liquid Hourly Space Velocity (OLHSV) of 0.057 h⁻¹. To simulate the operation of a 5-stage multistage reactor configuration, alkylate produced in the 4-stage reactor from Example 8 was co-fed at a rate of 3.25 cc/hr. Average 2-butene conversion at 0.057 h⁻¹ was 58.1% at 10 days of co-feeding and continued to drop with days on stream. As demonstrated by this example, the presence of about 2% C5+ olefins in the feed caused the 2-butene conversion to decrease by about 41.6% as compared to example 10.

### Example 12

The catalyst of Example 2 was loaded into a pilot plant single stage reactor, as shown in FIG. 1A. The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor bed. The alkylation product mixture was measured using a FID GC equipped with a 150 m Petrocol column. The flow to the reactor was set to achieve an Olefin Liquid Hourly Space Velocity (OLHSV) of 0.057 To simulate the operation of a 5-stage multistage reactor configuration where the heavier olefin content has been reduced by hydrogenation, the hydrogenated alkylate prepared in Example 9 was co-fed at a rate of 3.25 cc/hr. Average 2-butene conversion at 0.057 h⁻¹ was 98.8%. As demonstrated by this example, the presence of about 0.15% C5+ olefins in the feed caused the 2-butene conversion to decrease by less than 1% vs. the reference case in Example 10.

### Example 13

The catalyst of Example 4 was loaded into a pilot plant single-stage reactor, as shown in FIG. 1A. The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was varied from 450 psig to 950 psig (3103 kPag to 6550 kPag), corresponding to an isobutane density of 79-354 kg/m³ at 150° C. A gas blend prepared by mixing isobutane and 2-butene at an i:o ratio of 40:1 was fed to the top of the reactor bed. The reactor effluent was measured using a FID GC equipped with a 150 m Petrocol column. The flow to the reactor was set to achieve an Olefin Liquid Hourly Space Velocity (OLHSV) of 0.057 h⁻¹. Conversion of 2-butene as a function of pressure is shown in the Table 1 below (selectivity data is in g/g C5+):

TABLE 1

| | Conversion | Product Selectivity | | | |
|---|---|---|---|---|---|
| Pressure, psig | 2-Butene | C8 Paraffin | C8 Olefin | C9+ | C7− |
| 450 | 60.9 | 36.4 | 24.3 | 21.8 | 11.5 |
| 550 | 74.0 | 39.5 | 19.4 | 19.4 | 13.6 |
| 750 | 94.3 | 44.1 | 13.5 | 23.5 | 16.4 |
| 850 | 96.3 | 45.1 | 12.6 | 22.2 | 17.7 |
| 950 | 97.1 | 45.9 | 12.4 | 20.2 | 19.4 |

### Example 14

The catalyst of Example 4 was loaded into a pilot plant four-stage reactor, as shown in FIG. 1C. Each stage was 60″ long and made from ¾″ O.D. Schedule 40 pipe. Each stage was loaded with 150 g of catalyst. The reactor was located in an isothermal sand bath maintained at varied temperatures from 130° C. to 160° C. Reactor pressure was 750 psig to 850 psig (5171 kPag to 5860 kPag). Isobutane (99.6% purity) was fed to the first reactor bed and the propylene flow was split evenly into 4 using Coriolis meters and independently fed to each reactor stage. The relative rates of isobutane and propylene were set such that the isobutane to propylene ratio at the top of the first stage was about 80:1. The alkylation product mixture exiting the reactor was measured using a FID GC equipped with a 150 m Petrocol column. The total propylene flow to the reactor was set to achieve an Olefin Liquid Hourly Space Velocity (OLHSV) of 0.049 h⁻¹.

Average propylene conversion as a function of reactor conditions is shown in Table 2 below. The alkylation product mixture was sent to a distillation column for separation of the C4-hydrocarbons and the C5+ portion of the alkylation product mixture was analyzed via an off-line FID GC equipped with a 150 m Petrocol column. C8 and C9+ olefin contents were determined and are also shown in Table 2 below.

TABLE 2

| | Pressure | Conversion | C8 | C9+ | |
|---|---|---|---|---|---|
| Temperature | (psig) | (%) | Olefin | Olefin | C8 |
| 130 | 750 | 95.9 | 1.97 | 1.6 | 1.23 |
| 140 | 850 | 98.1 | 1.81 | 1.4 | 1.25 |
| 150 | 850 | 99.7 | 0.76 | 0.74 | 1.85 |
| 160 | 850 | 100 | 0.61 | 0.55 | 1.91 |

As shown in examples above, the production of undesired olefins in the alkylation reactor leads to a nonlinear increase of heavier fraction in the alkylate products in the subsequent stages and leads to catalyst deactivation. The examples in this disclosure show the reduction in undesired heavies as well as an increase in the desired products can be achieved by increasing the isoparaffin partial pressure in the reactor and by operating the reactors at increased temperature compared to previous processes.

Overall, it has been discovered that certain byproducts, including olefin oligomers, produced during the alkylation of isoparaffins with olefins in a multistage reactor may decrease catalyst activity and reduce conversion of olefins. It has also been discovered that the production of olefin oligomers may be reduced or eliminated by increasing the i:o ratio to about 100:1 or greater, increasing the reactor pressure, and/or increasing the reactor temperature. A multistage reactor may provide greater conversion and production rates and decrease overall costs of production. The combination of using a reactor having two or more stages and one or more of i) increased i:o ratio of about 100:1 or greater, ii) increased reactor pressure, or iii) increased reactor temperature, which may provide reduced production of olefin oligomers, increased olefin conversion, increased production, decreased catalyst deactivation, and/or improved product selectivity, as compared to previous solid acid alkylation processes.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

The invention claimed is:

1. A process for the alkylation of an isoparaffin, the process comprising:
introducing, in a multistage reactor, a solid acid catalyst to an isoparaffin feed and an olefin feed at a pressure of about 300 psig to about 1500 psig to form an alkylation product mixture;
wherein the solid acid catalyst comprises a crystalline microporous material of the MWW framework type.

2. The process of claim 1, wherein the pressure is about 450 psig or greater.

3. The process of claim 1, wherein the pressure is about 750 psig or greater.

4. The process of claim 1, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or combination(s) thereof.

5. The process of claim 1, wherein the isoparaffin feed comprises isobutane, isopentane, or combination(s) thereof.

6. The process of claim 1, wherein the olefin feed comprises one or more C2-05 olefins.

7. The process of claim 6, wherein the olefin feed comprises propene, 1-butene, 2-butene, isobutylene, or combination(s) thereof.

8. The process of claim 1, where the alkylation product mixture includes less than 5 wt % C8+ olefins

9. The process of claim 1, wherein introducing the solid acid catalyst to an isoparaffin feed and an olefin feed is performed at a ratio of isoparaffin:olefin of about 120:1 or greater.

10. A process for the alkylation of an isoparaffin, the process comprising:
introducing, in a multistage reactor, a solid acid catalyst to an isoparaffin feed and an olefin feed at a temperature of from about 100° C. to about 200° C. to form an alkylation product mixture,
wherein the solid acid catalyst comprises a crystalline microporous material of the MWW framework type.

11. The process of claim 10, wherein the temperature is about 130° C. or greater.

12. The process of claim 10, wherein the temperature is about 140° C. or greater.

13. The process of claim 10, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or combination(s) thereof.

14. The process of claim 10, wherein the isoparaffin feed comprises isobutane.

15. The process of claim 14, wherein contacting the solid acid catalyst with an isoparaffin feed and an olefin feed is performed at a pressure and a temperature greater than the critical point of isobutane.

16. The process of claim 10, wherein the isoparaffin feed comprises isopentane.

17. The process of claim 16, wherein contacting the solid acid catalyst with an isoparaffin feed and an olefin feed is performed at a pressure and a temperature greater than the critical point of isopentane.

18. The process of claim 10, wherein the olefin feed comprises propene, 2-butene, 1-butene, or combination(s) thereof.

19. The process of claim 10, wherein the alkylation product mixture comprises less than 5 wt % C8+ olefins.

20. The process of claim 10, wherein introducing the solid acid catalyst to an isoparaffin feed and an olefin feed is performed at a ratio of isoparaffin:olefin of about 120:1 or greater.
